(19) United States
(12) Patent Application Publication
(10) Pub. No.: US 2010/0305523 A1
(43) Pub. Date: Dec. 2, 2010

(54) Active Exudate Control System

(75) Inventor: Mark A. Vess, Hanson, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/472,714

(22) Filed: May 27, 2009

(51) Int. Cl. A61M 1/00; A61F 13/00

(52) U.S. Cl. 604/313

(57) ABSTRACT

A portable negative pressure wound therapy system includes a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister has ports to introduce a vacuum from the vacuum source into the collection canister. A tip sensor is provided to detect a change in an orientation of the canister assembly. The canister assembly also includes valve assemblies, each valve assembly corresponds to one of the ports. The controller communicates with each valve assembly to selectively open or close the port corresponding to the valve assembly based on an output from the tip sensor.

### BACKGROUND

1. Technical Field

The present disclosure relates generally to treating a wound by applying negative pressure to the wound, and, more specifically, to a portable negative pressure wound therapy system operable to select a number of vacuum ports away from the exudate contained within a canister.

2. Description of Related Art

Wound closure involves the migration of epithelial and subcutaneous tissue adjacent the wound towards the center and away from the base of the wound until the wound closes. Unfortunately, closure is difficult with large wounds, chronic wounds or wounds that have become infected. In such wounds, a zone of stasis (i.e. an area in which localized swelling of tissue restricts the flow of blood to the tissues) forms near the surface of the wound. Without sufficient blood flow, the epithelial and subcutaneous tissues surrounding the wound not only receive diminished oxygen and nutrients, but, are also less able to successfully fight microbial infection and, thus, are less able to close the wound naturally. Such wounds have presented difficulties to medical personnel for many years.

Negative pressure wound therapy (NPWT), also known as suction or vacuum therapy, has been used in treating and healing wounds. Application of negative pressure, e.g. reduced or sub-atmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

Generally, negative pressure therapy provides for a wound covering to be positioned over the wound to facilitate suction at the wound area. A conduit is introduced through the wound covering to provide fluid communication to an external vacuum source. Atmospheric gas, wound exudates, or other fluids may thus be drawn from the reservoir through the fluid conduit to stimulate healing of the wound. Exudates drawn from the reservoir may be deposited in a collection canister.

Often, a portable NPWT device is worn by the patient so that the patient may remain ambulatory instead of being confined to a stationary position. While a patient is ambulatory, the portable NPWT device tends to tip or tilt in a multitude of directions. If there are enough exudates in the collection canister, the exudates may cover a suction port leading from the vacuum source to the collection canister because fluid seeks its own level. Covering the suction port prevents the application of negative pressure to the wound thereby discontinuing wound therapy. Additionally, covering the suction port may provide a false indication that the collection canister is full and needs to be replaced when there may be additional space in the canister to fill with exudate.

In addition, portable NPWT devices have a control unit attached to the canister. The control unit generally contains the suction pump and sensitive electronics such as a pressure transducers, microprocessors, or the like. When the NPWT device tips, exudate may aspirate from the canister into the control unit thereby damaging the suction pump and/or electronic components.

### SUMMARY

The present disclosure relates to a portable NPWT system including a dressing assembly for positioning over a wound to apply a negative pressure to the wound and a canister assembly. The canister assembly includes a control unit having a vacuum source and a controller and a collection canister in communication with the dressing assembly operable to receive fluid from the wound. The collection canister has ports to introduce a vacuum from the vacuum source into the collection canister. A tip sensor is provided to detect a change in an orientation of the canister assembly. The canister assembly also includes valve assemblies, each valve assembly corresponds to one of the ports. The controller communicates with each valve assembly to selectively open or close the port corresponding to the valve assembly based on an output from the tip sensor.

### BRIEF DESCRIPTION OF THE DRAWINGS

Various embodiments of the wound dressing system of the present disclosure are described herein with reference to the drawings wherein:

FIG. 1 is a diagram of an embodiment of a NPWT system in accordance with the present disclosure;

FIG. 2 is a diagram of an embodiment of a NPWT system in accordance with the present disclosure;

FIG. 3 is a diagram of an embodiment of a NPWT system in accordance with the present disclosure;

FIG. 4 is a diagram of an embodiment of a NPWT system, in accordance with the present disclosure;

FIG. 5 is a diagram of an embodiment of a NPWT system, in accordance with the present disclosure; and

FIG. 6 is a diagram of an embodiment of a NPWT system, in accordance with the present disclosure.

### DESCRIPTION OF THE PREFERRED EMBODIMENTS

Hereinafter, embodiments of the presently disclosed NPWT systems will be described with reference to the accompanying drawings. Like reference numerals may refer to similar or identical elements throughout the description of the figures. As used herein, “wound exudate”, or, simply, “exudate”, generally refers to any fluid output from a wound, e.g., blood, serum, and/or pus, etc. As used herein, “fluid” generally refers to a liquid, a gas or both.

Various embodiments of the present disclosure provide NPWT systems (or apparatus). Embodiments of the presently disclosed NPWT systems are generally suitable for use in applying negative pressure to a wound to facilitate healing of the wound in accordance with various treatment modalities.

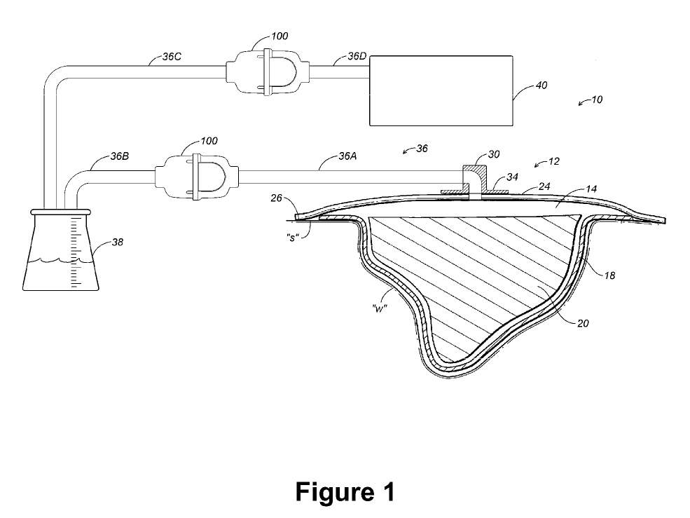

Referring to FIG. 1, a NPWT system according to an embodiment of the present disclosure is depicted generally as 10 for use on a wound bed “w” surrounded by healthy skin “s”. NPWT system 10 includes a wound dressing 12 positioned relative to the wound bed “w” to define a vacuum chamber 14 about the wound bed “w” to maintain negative pressure at the wound area. Wound dressing 12 includes a contact layer 18, a wound filler 20 and a wound cover 24.

Contact layer 18 is intended for placement within the wound bed “w” and may be relatively non-supportive or flexible to substantially conform to the topography of the wound bed “w”. A variety of materials may be used for the contact layer 18. Contact layer 18 selection may depend on various factors such as the patient's condition, the condition of the periwound skin, the amount of exudate and/or the condition of the wound bed “w”. Contact layer 18 may be formed from perforated film material. The porous characteristic of the contact layer 18 permits exudate to pass from the wound bed “w” through the contact layer 18. Passage of wound exudate through the contact layer 18 may be substantially unidirectional such that exudate does not tend to flow back into the wound bed “w”. Unidirectional flow may be encouraged by directional apertures, e.g., apertures positioned at peaks of undulations or cone-shaped formations protruding from the contact layer 18. Unidirectional flow may also be encouraged by laminating the contact layer 18 with materials having absorption properties differing from those of the contact layer 18, or by selection of materials that promote directional flow. A non-adherent material may be selected for forming the contact layer 18 such that the contact layer 18 does not tend to cling to the wound bed “w” or surrounding tissue when it is removed. One example of a material that may be suitable for use as a contact layer 18 is commercially available under the trademark XEROFLOW® offered by Tyco Healthcare Group LP (d/b/a Covidien). Another example of a material that may be suitable for use as the contact layer 18 is the commercially available CURITY® non-adherent dressing offered by Tyco Healthcare Group LP (d/b/a Covidien).

Wound filler 20 is positioned in the wound bed “w” over the contact layer 18 and is intended to transfer wound exudate. Wound filler 20 is conformable to assume the shape of any wound bed “w” and may be packed up to any level, e.g., up to the level of healthy skin “s” or to overfill the wound such that wound filler 20 protrudes over healthy skin “s”. Wound filler 20 may be treated with agents such as polyhexamethylene biguanide (PHMB) to decrease the incidence of infection and/or other medicaments to promote wound healing. A variety of materials may be used for the wound filler 20. An example of a material that may be suitable for use as the wound filler 20 is the antimicrobial dressing commercially available under the trademark KERLIX™ AMD™ offered by Tyco Healthcare Group LP (d/b/a Covidien).

Cover layer 24 may be formed of a flexible membrane, e.g., a polymeric or elastomeric film, which may include a biocompatible adhesive on at least a portion of the cover layer 24, e.g., at the periphery 26 of the cover layer 24. Alternately, the cover layer 24 may be a substantially rigid member. Cover layer 24 may be positioned over the wound bed “w” such that a substantially continuous band of a biocompatible adhesive at the periphery 26 of the cover layer 24 forms a substantially fluid-tight seal with the surrounding skin “s”. Cover layer 24 may act as both a microbial barrier and a fluid barrier to prevent contaminants from entering the wound bed “w” and to help maintain the integrity thereof.

In one embodiment, the cover layer 24 is formed from a moisture vapor permeable membrane, e.g., to promote the exchange of oxygen and moisture between the wound bed “w” and the atmosphere. An example of a membrane that may provide a suitable moisture vapor transmission rate (MVTR) is a transparent membrane commercially available under the trade name POLYSKIN®II offered by Tyco Healthcare Group LP (d/b/a Covidien). A transparent membrane may help to permit a visual assessment of wound conditions to be made without requiring removal of the cover layer 24.

Wound dressing 12 may include a vacuum port 30 having a flange 34 to facilitate connection of the vacuum chamber 14 to a vacuum system. Vacuum port 30 may be configured as a rigid or flexible, low-profile component and may be adapted to receive a conduit 36 in a releasable and fluid-tight manner. An adhesive on at least a portion of the underside of the flange 34 may be used to provide a mechanism for affixing the vacuum port 30 to the cover layer 24. The relative positions, size and/or shape of the vacuum port 30 and the flange 34 may be varied from an embodiment depicted in FIG. 1. For example, the flange 34 may be positioned within the vacuum chamber 14 such that an adhesive on at least a portion of an upper side surface of the flange 34 affixes the vacuum port 30 to the cover layer 24. A hollow interior portion of the vacuum port 30 provides fluid communication between the conduit 36 and the vacuum chamber 14. Conduit 36 extends from the vacuum port 30 to provide fluid communication between the vacuum chamber 14 and the vacuum source 40. Alternately, the vacuum port 30 may not be included in the dressing 12 if other provisions are made for providing fluid communication with the conduit 36.

Any suitable conduit may be used for the conduit 36, including conduit fabricated from flexible elastomeric or polymeric materials. In the NPWT system 10 illustrated in FIG. 1, the conduit 36 includes a first conduit section 36A, a second conduit section 36B, a third conduit section 36C and a fourth conduit section 36D. The first conduit section 36A extends from the vacuum port 30 and is coupled via a fluid line coupling 100 to the second conduit section 36B, which extends to the collection canister 38. The third conduit section 36C extends from the collection canister 38 and is coupled via another fluid line coupling 100 to the fourth conduit section 36D, which extends to the vacuum source 40. The shape, size and/or number of conduit sections of the conduit 36 may be varied from the first, second, third and fourth conduit sections 36A, 36B, 36C and 36D depicted in FIG. 1.

The first, second, third and fourth conduit sections 36A, 36B, 36C and 36D of the conduit 36 may be connected to components of the system 10 by conventional air-tight means, such as, for example, friction fit, bayonet coupling, or barbed connectors. The connections may be made permanent. Alternately, a quick-disconnect or other releasable connection means may be used to provide some adjustment flexibility to the system 10.

Collection canister 38 may be formed of any type of container that is suitable for containing wound fluids. For example, a semi-rigid plastic bottle may be used for the collection canister 38. A flexible polymeric pouch or other hollow container body may be used for the collection canister 38. Collection canister 38 may contain an absorbent material to consolidate or contain the wound fluids or debris. For example, super absorbent polymers (SAP), silica gel, sodium polyacrylate, potassium polyacrylamide or related compounds may be provided within collection canister 38. At least a portion of canister 38 may be transparent or semi-transparent, e.g., to permit a visual assessment of the wound exudate to assist in evaluating the color, quality and/or quantity of exudate. A transparent or semi-transparent portion of the collection canister 38 may permit a visual assessment to assist in determining the remaining capacity or open volume of the canister and/or may assist in determining whether to replace the collection canister 38.

The collection canister 38 is in fluid communication with the wound dressing 12 via the first and second conduit sections 36A, 36B. The third and fourth conduit sections 36C, 36D connect the collection canister 38 to the vacuum source 40 that generates or otherwise provides a negative pressure to the collection canister 38. Vacuum source 40 may include a peristaltic pump, a diaphragmatic pump or other suitable mechanism. Vacuum source 40 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg to about 125 mmHg, or about 40 mmHg to about 80 mmHg. One example of a peristaltic pump that may be used as the vacuum source 40 is the commercially available Kangaroo PET Eternal Feeding Pump offered by Tyco Healthcare Group LP (d/b/a Covidien).

In embodiments, the NPWT system 10 includes one or more fluid line couplings 100 that allow for selectable coupling and decoupling of conduit sections. For example, a fluid line coupling 100 may be used to maintain fluid communication between the first and second conduit sections 36A, 36B when engaged, and may interrupt fluid flow between the first and second conduit sections 36A, 36B when disengaged. Thus, fluid line coupling 100 may facilitate the connection, disconnection or maintenance of components of the NPWT system 10, including the replacement of the collection canister 38. Additional or alternate placement of one or more fluid line couplings 100 at any location in line with the conduit 36 may facilitate other procedures. For example, the placement of a fluid line coupling 100 between the third and fourth conduit sections 36C, 36D, as depicted in FIG. 1, may facilitate servicing of the vacuum source 40.

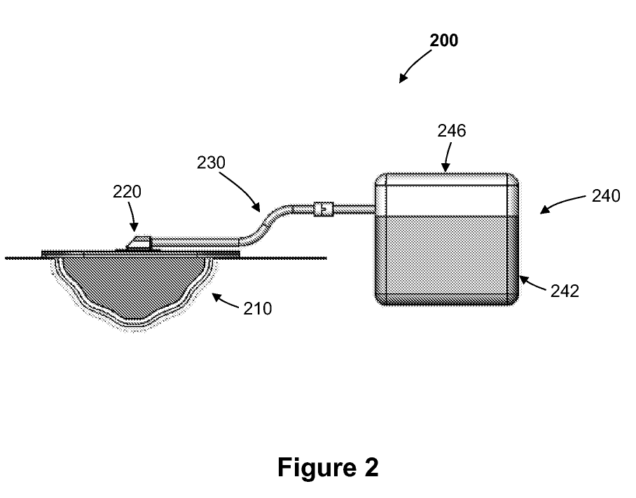

Referring to FIG. 2, the portable NPWT system shown generally as 200 includes a dressing assembly 210, a wound port assembly 220, an extension assembly 230 and a canister assembly 240. Dressing assembly 210 is positioned relative to the wound area to define a vacuum chamber about the wound area to maintain negative pressure at the wound area. Dressing assembly 210 may be substantially sealed from extraneous air leakage, e.g., using adhesive coverings. Wound port assembly 220 is mounted to the dressing assembly 210. For example, wound port assembly 220 may include a substantially continuous band of adhesive at its periphery for affixing the wound port assembly 220 to the dressing assembly 210. Extension assembly 230 is coupled between the wound port assembly 220 and the canister assembly 240 and defines a fluid flow path between the wound port assembly 220 and the canister assembly 240. A hollow interior of the wound port assembly 220 provides fluid communication between the extension assembly 230 and the interior of the dressing assembly 210. Dressing assembly 210 and the wound port assembly 220 shown in FIG. 2 are similar to components of the wound dressing 12 of FIG. 1 and further description thereof is omitted in the interests of brevity.

Canister assembly 240 includes a control unit 246 and a collection canister 242 disposed below the control unit 246. Control unit 246 and the collection canister 242 may be releasably coupled. Mechanisms for selective coupling and decoupling of the control unit 246 and the collection canister 242 include fasteners, latches, clips, straps, bayonet mounts, magnetic couplings, and other devices. Collection canister 242 may comprise any container suitable for containing wound fluids.

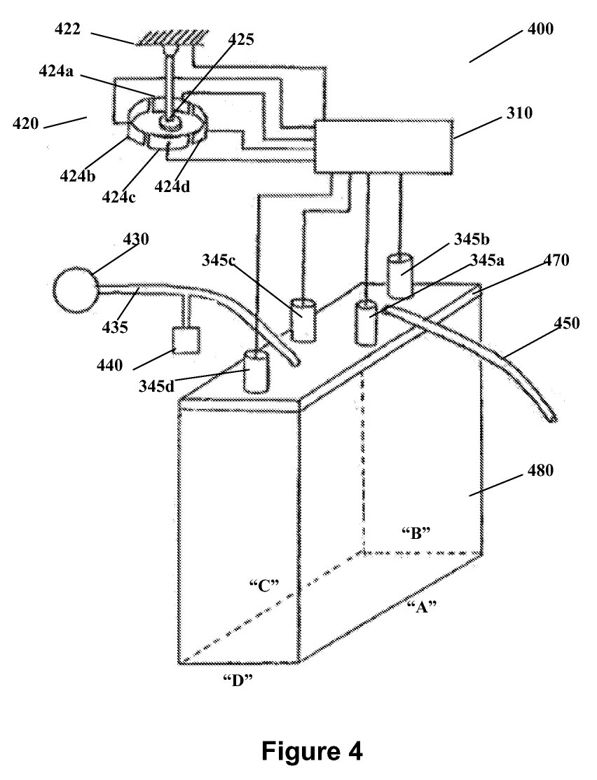

In one embodiment, the portable NPWT system 200 is capable of operating in a continuous mode or an alternating mode. In the continuous mode, the control unit 246 controls a pump (e.g., suction pump 430 shown in FIG. 4) to continuously supply a selected vacuum level at the collection canister 242 to create a reduced pressure state within the dressing assembly 210. In the alternating mode, the control unit 246 controls the pump to alternating supply a first negative pressure, e.g., about 80 mmHg, at the collection canister 242 for a preset fixed amount of time and a second negative pressure, e.g., about 50 mmHg, at the collection canister 242 for a different preset fixed amount of time. In general, the output of the pump is directly related to the degree of air leakage in the portable NPWT system 200 and the open volume in the collection canister 242. If there is sufficient air leakage in the system 200, e.g., at the dressing assembly 210, the pump can remain on continuously and the control unit 246 can control negative pressure at the collection canister 242 by adjusting the pump speed. Alternatively, if there is not sufficient air leakage in the system 200 to permit the pump to remain on continuously, the control unit 246 can control negative pressure at the collection canister 242 by turning the pump on and off, e.g., for non-equal on/off periods of time.

If an air leak develops in the portable NPWT system 200, e.g., at the dressing assembly 210, for which the control unit 246 can not compensate by increasing the pump speed, the control unit 246 may indicate an alarm. For example, the control unit 246 may indicate a leak alarm after two consecutive minutes of operation in which the vacuum level is below the current set point (or below the minimum level of a set point range). Audible indicatory means may also be incorporated or associated with the control unit 246 to notify the user of a condition, e.g., leak, occlusion or system error conditions.

In embodiments, the control unit 246 includes a user interface (not shown) and a printed circuit board (PCB) (not shown). The PCB includes a microprocessor (not shown). A pressure transducer (e.g., transducer 440 shown in FIG. 4) is electrically coupled to the PCB.

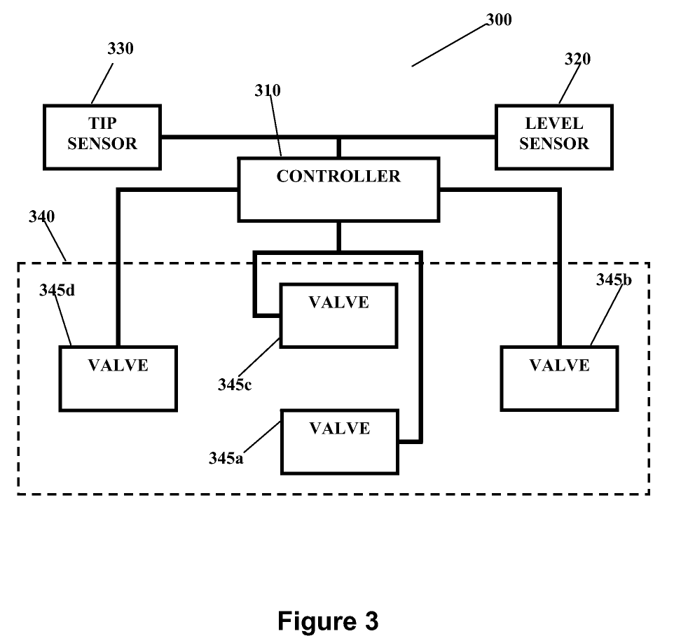

Referring to FIG. 3, the portable NPWT system has a valve control system shown generally as 300. The valve control system 300 includes a controller 310, a level sensor 320, a tip sensor 330, and valve assemblies 345a, 345b, 345c and 345d. Controller 310 may include at least one processor (not shown) and at least one memory module (not shown). The memory module may be a volatile memory (e.g. DRAM, SRAM, or the like) or a non-volatile memory (e.g., ROM, PROM, EPROM, EEPROM, a semiconductor flash memory, or the like). The memory module stores instructions for controlling the valves based on a signal received from the level sensor 320 and/or tip sensor 330. When a signal is received from the level sensor 320 and/or tip sensor 330, the processor of controller 310 executes the instructions stored in the memory module to control the valves.

Level sensor 320 determines if the canister assembly 240, as shown in FIG. 2, is inverted. Level sensor 320 may be incorporated into the control unit 246 or the collection canister 242. The embodiments described in the present disclosure are not limited to any particular level sensor. The level sensor 320 may be float level sensor, conductive or electrode based level sensor, capacitance level sensors, optical interface level sensors, ultrasonic level sensors, magnetostrictive level sensors, resistive chain level sensors, air bubbler level systems, or any other sensor that may be used to measure the exudate level. (Are there any other level sensors we should add to the list? Are there any level sensors we should remove? Which level sensor is most appropriate to be included in the canister assembly?) When the level sensor 320 determines that the canister assembly is inverted, a signal is transmitted to controller 310. Controller 310 ceases all signals to the valves so that the valves remain closed. In addition, the pump ceases operation when the canister assembly 240 is inverted.

Tip sensor 330 determines the orientation of the canister assembly while a patient is ambulatory. Tip sensor 330 may be a four position angle sensor which will be described hereinbelow with reference to FIG. 4. Alternatively, in another embodiment, tip sensor 330 may include a pair of accelerometers (not shown). The accelerometers may be placed in a perpendicular arrangement to detect a tip or tilt in two directions. When the accelerometers detect a tip or tilt, the accelerometers provide a signal to the controller 310 to selectively operate the valve assemblies.

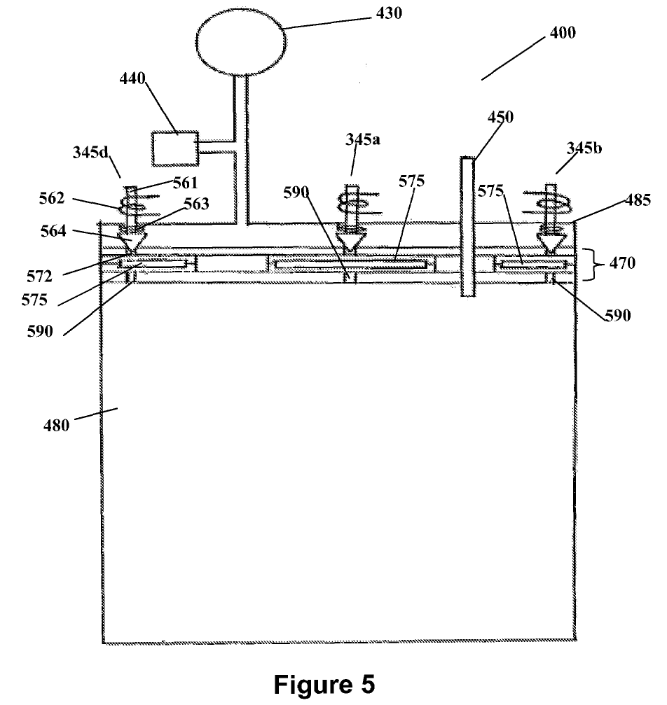

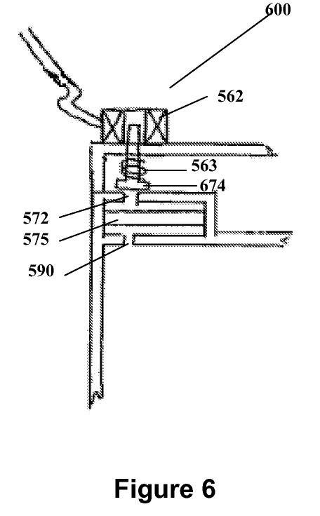

Valve assemblies 345a, 345b, 345c and 345d are arranged in a substantially rectangular plane shown generally as 340. Embodiments of the valve assemblies will be described in detail hereinbelow with reference to FIG. 5 and FIG. 6B. As shown in FIG. 3, the valve assemblies are arranged in a cross like pattern with each valve assembly being located in a central position along a side of the rectangular plane 340. Although FIG. 3 depicts a rectangular plane 340 with four valve assemblies and number of shapes can be used for the plane as well as any number valve assemblies may be used. For instance, instead of four valve assemblies being used in the rectangular plane, eight valve assemblies can be situated on the plane with a valve assembly at each corner and four valve assemblies positioned as shown in FIG. 3. Alternatively, instead of a rectangular plane, a circular plane may be employed where the valve assemblies are positioned at equal intervals along the circumference of the circle.

Referring to FIG. 4, a canister assembly according to an embodiment of the present disclosure is shown generally as 400. Canister assembly 400 includes a canister 480 that is similar to collection canister 242 shown in FIG. 2. Suction pump 430 provides a source of negative or reduced pressure to the canister 480 through conduit 435. A pressure transducer 440 is in communication with conduit 435 to measure the pressure in conduit 435. Exudate travels from the wound “w” through exudate conduit 450 into the canister 480. At the top of canister 480 is a canister interface 470 which will be described in detail with reference to FIG. 5.

Canister assembly 400 has a controller 310 that receives a signal from the tip sensor 420 to control valve assemblies 345a, 345b, 345c and 345d. Tip sensor 420 is tip or tilt switch having a center contact 422. A pendulum 425 is electrically coupled to center contact 422. Surrounding the base of the pendulum 425 are four contacts 424a, 424b, 424c and 424d.

Operation of the canister assembly 400 when the canister assembly is tipped or tilted will be described with reference to FIG. 4. When the canister 480 is tipped in the direction of side “A”, pendulum 425 makes contact with contact 424a. Such contact provides a signal to the controller 310 which keeps valve assembly 345a open and closes the other three valves. When the canister 480 is tipped in the direction of side “B”, pendulum 425 makes contact with contact 424b. Such contact provides a signal to the controller 310 which keeps valve assembly 345b open and closes the other three valves. When the canister 480 is tipped in the direction of side “C”, pendulum 425 makes contact with contact 424c. Such contact provides a signal to the controller 310 which keeps valve assembly 345c open and closes the other three valves. When the canister 480 is tipped in the direction of side “D”, pendulum 425 makes contact with contact 424d. Such contact provides a signal to the controller 310 which keeps valve assembly 345d open and closes the other three valves. When the pendulum does not make contact with any of contacts 424a, 424b, 424c and 424d all the valve assemblies remain open

As exudate fills the canister, which valve assemblies remain open tend to shift more open. The shift between valve assemblies can be measured. When such shifting between valve assemblies reaches a threshold, the NPWT device can recognize such rapid shifting a full or replace canister condition.

Referring to FIG. 5, the valve assembly and canister interface of canister assembly 400 are shown in more detail. Valve assembly 345d has a rod 561 connected to a plunger head 564. Wrapped around rod 561 and situated in between plunger head 564 and base 485 is a spring 563. Rod 561 passes through a solenoid coil 562. Rod 561 is a metallic object that moves when it is subjected to an electromagnetic force. Rod 561 is made from (please insert materials here). Plunger 564 is made from silicone, plastic or other rubber like material. When a voltage is applied to solenoid coil 562, rod 561 is moved from its stationary position to an active position thereby compressing spring 563 and opening a passageway in the canister interface 470 to allow a negative or reduced pressure to be applied to the canister 480. Valve assemblies 345a, 345b and 345c are constructed in a similar manner as valve assembly 345d.

Canister interface 470 has a vacuum port 572 and a canister port 590. In between vacuum port 572 and canister port 590 is a hydrophobic filter 575. Hydrophobic filter 575 helps prevent exudate from aspirating into the chamber above the canister interface 470 and into the suction pump 430. Hydrophobic filter 575 may be a replaceable cartridge that can be replaced during a canister change. Hydrophobic filter 575 may also include an antibacterial coating. Although not shown, an odor filter such as a charcoal filter may also be placed in canister interface 470 in line with the hydrophobic filter to reduce odor produced by exudate.

Referring to FIG. 6, another embodiment of a valve assembly is generally shown as 600. As shown in valve assembly 600, a solenoid poppet is used to close the vacuum port. When there is no voltage applied to the solenoid coil 562, the spring 563 is in a decompressed state thereby closing the vacuum port 572 with poppet 674. When a voltage is applied to the solenoid coil 562, the poppet 674 moves to an active position and spring 563 is compressed until the voltage is no longer applied.

The components of the canister assembly described above can have a variety of arrangements while still rendering the NPWT device operational. For instance, the valve assemblies may be located in the control unit 246 or in the collection canister 242. Similarly, canister interface 470 may be located in either the control unit 246 or the collection canister 242. Although the valve assembly was described above as having a plunger or a poppet, the valve assembly could be as simple as a ball bearing that is held on the vacuum port by a plastic flap and is released from its seat by an overhead coil when activated.

While the disclosure has been illustrated and described, it is not intended to be limited to the details shown, since various modifications and substitutions can be made without departing in any way from the spirit of the present disclosure. As such, further modifications and equivalents of the invention herein disclosed can occur to persons skilled in the art using no more than routine experimentation, and all such modifications and equivalents are believed to be within the spirit and scope of the disclosure as defined by the following claims.

What is claimed is:

1. A portable negative pressure wound therapy system comprising:
a dressing assembly for positioning over a wound to apply a negative pressure to the wound; and
a canister assembly including:
a control unit having a vacuum source and a controller;
a collection canister in communication with the dressing assembly operable to receive fluid from the wound, the collection canister having a plurality of ports to introduce a vacuum from the vacuum source into the collection canister;
a tip sensor operable to detect a change in an orientation of the canister assembly; and
a plurality of valve assemblies, each valve assembly corresponds to one of the plurality of ports,
the controller communicates with each valve assembly to selectively open or close the port corresponding to the valve assembly based on an output from the tip sensor.

2. The portable negative pressure wound therapy system according to claim 1, wherein the canister assembly further includes a canister interface, the canister interface is positioned between the control unit and the collection canister.

3. The portable negative pressure wound therapy system according to claim 2, wherein the plurality of valve assemblies are positioned at the canister interface.

4. The portable negative pressure wound therapy system according to claim 2, wherein the canister interface further includes a plurality of filters with each filter corresponding to a different valve assembly.

5. The portable negative pressure wound therapy system according to claim 3, wherein each valve assembly is positioned at the center of each edge of the canister interface.

6. The portable negative pressure wound therapy system according to claim 1, wherein each valve assembly includes a plunger member, a spring and a solenoid coil.

7. The portable negative pressure wound therapy system according to claim 1, wherein each valve assembly includes a poppet member, a spring and a solenoid coil.

8. The portable negative pressure wound therapy system according to claim 1, wherein the tip sensor includes a four-position angle sensor.

9. The portable negative pressure wound therapy system according to claim 1, wherein the tip sensor includes a plurality of accelerometers.

10. The portable negative pressure wound therapy system according to claim 1, further comprising a level sensor, the level sensor is operable to detect an inversion of the canister assembly and output a signal to the controller when the canister assembly is inverted, the controller controls each valve assembly to close the port corresponding to the valve assembly when the controller receives the signal from the level sensor.
